(19) United States
(12) Patent Application Publication
(10) Pub. No.: US 2005/0159490 A1
(43) Pub. Date: Jul. 21, 2005

(54) Method to treat chronic heart failure and/or elevated cholesterol levels

(76) Inventor: Eugene Morkin, Tucson, AZ (US)

Correspondence Address: HAYES, SOLOWAY P.C., 130 W. CUSHING STREET, TUCSON, AZ, 85701, US

(21) Appl. No.: 10/818,541

(22) Filed: Apr. 5, 2004

Related U.S. Application Data
(63) Continuation-in-part of application No. 10/368,755, filed on Feb. 18, 2003, now Pat. No. 6,716,877.
Application No. 10/368,755 is a continuation-in-part of application No. 09/774,994, filed on Jan. 31, 2001, now Pat. No. 6,534,676.

(51) Int. Cl. A61K 31/198

(52) U.S. Cl. 514/567; 424/423; 424/451

(57) ABSTRACT

A method for treating a patient having congestive heart failure by administering a therapeutically effective amount of a thyroid hormone analog sufficient to produce an increase in cardiac index of at least 15% while increasing heart rate no more than 10 beats per minute.

### CROSS REFERENCE TO RELATED APPLICATION

This application is a Continuation-in-Part of co-pending U.S. application Ser. No. 10/368,755, filed Feb. 18, 2003, which is, in turn, a continuation-in-part of U.S. application Ser. No. 09/774,994, filed Jan. 31, 2001, now U.S. Pat. No. 6,534,676, issued Mar. 18, 2003.

### FIELD OF THE INVENTION

The present invention relates to a treatment for patients having congestive heart failure and/or elevated cholesterol blood levels.

### BACKGROUND OF THE INVENTION

Congestive heart failure continues to be a major health problem, affecting about 4.6 million people in the United States, and its prevalence is predicted to increase over the next several decades. The magnitude of heart failure as a clinical problem has placed emphasis on the need to develop new treatment strategies.

One approach that has emerged is the use of thyroid hormone, which has unique physiologic and biochemical actions that make it a novel and potentially useful agent for treatment of heart failure. Thyroid hormone has been shown to act at the transcriptional level on the content of myocardial calcium cycling proteins to stimulate calcium uptake by sarcoplasmic reticulum. In addition, thyroid hormone causes a reciprocal shift in cardiac myosin heavy chain (MHC) isoform expression, increasing the expression of the high activity V₁ isoform and decreasing the low activity V₃ form. These biochemical alterations may underlie the ability of thyroid hormone to increase the rates of ventricular pressure development and relaxation.

Thyroid hormones include the L-forms of thyroxine (3,5,3′5′-L-thyronine; hereinafter thyroxine or T₄) and triiodothyronine (3′,3,5-L-triiodothyrone; hereinafter triiodothyronine or T₃). 3′,5′,3-L-Triiodothyronine (hereinafter Reverse T₃ or r T₃), is a normal metabolite of T₄. T₄ is synthesized in the thyroid gland and is the circulating form of hormone found in plasma. Although small amounts of T₃ are synthesized by the thyroid gland, the majority is formed from the metabolism of thyroxine in peripheral tissues by the enzyme 5′-monodeiodinase. The molecular basis for the actions of thyroid hormones is though to be mediated through the binding of T₃ to chromatin-bound nuclear receptors. There are two major subtypes of the thyroid hormone receptor, TRα and TRβ, which are the products of two different genes. These genes are members of the c-erbA protooncogene family and are related to a large number of steroid and peptide hormone receptors collectively known as the steroid-thyroid hormone superfamily. The TR α and β subtypes are differentially expressed in various tissues.

Thyroxine, synthesized by methods such as described in U.S. Pat. No. 2,803,654, is the principle thyroid hormone in current clinical use. This is largely because of its long half-life of 6-7 days. Triiodothyronine, which is less strongly bound to plasma proteins and has a more rapid onset of action, is available for intravenous administration. However, T₃ has a relatively short half-life of two days or less.

Numerous studies have been carried out to synthesize thyroid hormone analogs that mimic the actions of the natural hormones. The objective of most of these efforts has been to develop thyromimetics that lower plasma cholesterol without adverse cardiac effects. A series of thyroxine analogs and methods of synthesis are described in U.S. Pat. No. 3,109,023.

Thyroid hormone agonists that are highly selective for the thyroid hormone receptor β subtype are described in U.S. Pat. No. 5,883,294. U.S. Pat. No. 5,284,971 describes a class of thyromimetics, which have the distinguishing characteristic of a sulfonyl bridge in the diphenyl core.

A more recent development has been the use of thyroid hormones for the treatment of cardiovascular compromise. A method for the treatment of patients with sudden (acute) cardiovascular compromise by administration of thyroid hormone is described in U.S. Pat. No. 5,158,978. The method teaches administration of T₄ and T₃ after cardiac arrest by injection into a vein, a central venous catheter, into the pulmonary circulation or directly into the heart.

Short-term intravenous administration of T₃ to patients with advanced congestive failure has been shown to improve cardiac output and decrease arterial vascular resistance. Oral administration of L-thyroxine also has been shown to improve cardiac performance and exercise capacity in patients with idiopathic dilated cardiomyopathy when given for two weeks and 3 months. Although the number of patients in these studies was small, the results were generally favorable and established the basis for further investigation into the safety and potential benefits of treatment of heart failure with thyroid hormone or thyroid hormone analogs.

In addition to its well-known chronotropic and inotropic actions on the heart, thyroid hormone decreases arterial resistance, venous resistance and venous compliance. The net effect of these changes is to increase cardiac output more than arterial pressure, resulting in decreased calculated arterial vascular resistance.

Because of potential adverse effects of thyroid hormone, such as metabolic stimulation and tachycardia, what is required are thyroid hormone analogs with fewer undesirable side effects. In my earlier U.S. Pat. No. 6,534,676, with Pennock, Bahl and Goldman, we describe the use of 3,5-diiodothyropropionic acid (DITPA), a thyroid hormone analog, for treating patients with congestive heart failure. Like thyroid hormone, DITPA binds to nuclear T₃ receptors of the c-erbA proto-oncogene family. DITPA has been shown to improve left ventricular (LV) performance in post-infarction experimental models of heart failure when administered alone or in combination with an angiotensin I-converting enzyme inhibitor, with approximately half of the chronotropic effect and less metabolic stimulation than L-thyroxine.

As reported in my aforementioned patent, when used in experimental models of heart failure, DITPA acts similarly to thyroid hormone, affecting both the heart and the peripheral circulation. Loss of the normal increase in contractility with heart rate, referred to as the positive force-frequency relationship, has been reported both in failing human myocardium and in animal models of heart failure. DITPA administration prevents the flattened contraction-frequency relationship in single myocytes from infarcted rabbit hearts. DITPA improves myocyte function, enhances calcium transport in the sarcoplasmic reticulum (SR) and prevents the down regulation of SR proteins associated with post-infarction heart failure in rabbits. In normal primates, DITPA enhances the in vivo force-frequency and relaxation-frequency relationships in a manner similar to thyroid hormone. DITPA is able to bring about these hemodynamic changes without increasing cardiac mass appreciably or adversely affecting ventricular dimensions. A morphometric analysis indicates that in post-infarction rats treated with DITPA there is an increase in capillary growth in the border zone around the infarct.

In my aforesaid co-pending application Ser. No. 10/368,755, I describe the two other DIPTA-like compounds having similar utility, i.e., for treating patients with congestive heart failure. More particularly, I describe two more of the iodination propionic derivatives, namely the triiodo derivative 3′,3,5-triiodothyropropionic acid (or “TRIPROP”) and the tetraiodo derivative, 3,5,3′,5′-tetraiodothyropropionic acid (or “TETRAPROP”) of DIPTA as having thyromimetic effects in experimental studies¹ and as being effective clinically in reducing serum cholesterol without increasing basal metabolic rate (BMR)². These properties make them similar to DITPA in terms of the ability to treat congestive heart failure.
¹Money W. L., Meltzer R. I., Feldman D., Rawson R. W.: The Effects of Various Thyroxine Analogues on Suppression of ¹³¹I Uptake by the Rat Thyroid, Endocrinology 64: 123-125 (1959); Stasilli N. R., Kroc R. L., Meltzer R. I.: Antigoitrogenic and Calorigenic Activities of Thyroxine Analogues in Rats, Endocrinology 64: 62-82 (1959).

2 Leeper R. D., Mead A. W., Money W. L., Rawson R. W.: Metabolic Effects and Therapeutic Applications of Triiodothyropropionic Acid, Clin Pharmacol Ther 2: 13-21, 1961; Hill S. R., Jr., Barker S. B., McNeil J. H., Tingley J. O., Hibbett L. L.: The Metabolic Effects of the Acetic and Propionic Acid Analogs of Thyroxine and Triiodothyronine. J. Clin. Invest. 39: 523-533, 1960.

### BRIEF DESCRIPTION OF THE INVENTION

Having demonstrated the utility of DIPTA, TRIPOP and TETRAPROP for treating patients with congestive heart failure, I have now concluded that other thyroid hormone analogs similarly may be useful for treating congestive heart failure. More particularly, I have determined that any thyroid hormone analog that produces an increased cardiac output with little or no increase in heart rate advantageously may be used for treating congestive heart failure. Preferably administration of the thyroid analog in accordance with the present invention produces an increase in cardiac index, i.e., cardiac output/body surface area of at least about 15% with an increase in heart rate of less than about 10 beats per minute. Alternatively, the performance criteria may be expressed as providing a reduction in systemic vascular resistance index (SVRI), i.e., cardiac output/mean arterial pressure/body surface area of at least about 15%. Thus, any thyroid analog that increases mean cardiac output without materially increasing heart rate advantageously may be employed in connection with the subject invention. Thus, any thyroid analog that provides a mean increase of cardiac output of at least about 15% with a increase in heart rate of less than about 10 beats per minute advantageously may be used for treating patients with congestive heart failure in accordance with the present invention.

### DETAILED DESCRIPTION OF THE PREFERRED EMBODIMENTS

In principal, a selective TRβ1 agonist might mediate lipid-lowering actions of the hormone without unwanted cardiac side effects. Some of the older analogs were reported to have selectivity for binding to TRβ1. For example, Triac has an affinity for TRβ1 that is two- or three-times greater than T₃.³ Several more recently synthesized compounds have greater selectivity. The structures of representative TRβ1 selective analogs are shown below: 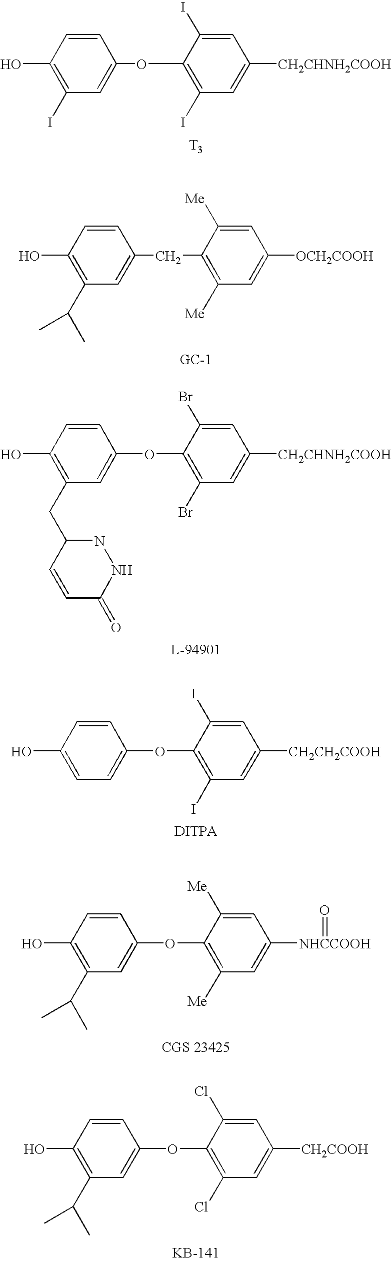
³Schueler P A, Schwartz H L, Strait K A, Mariash C N, Oppenheimer J I I. Binding of 3,5,3′-triiodothyronine (T₃) and its analogs to the in vitro translational products of c-erbA protooncogenes: Differences in the affinity of the α- and β-forms for the acetic acid analog and failure of the human testis and kidney α-2 products to bind T₃. 1990 Mol Endocrinol 4: 227-34.

The analog, {[3-isopropyl-4-hydroxyphenoxy]-3,5-dimethylphenyl]amino}-oxoacetate (CGS 23425), had a lower threshold for activation of TRβ1 than TRα1 in a transient transfection assay with an apoAI reporter construct. The concentration required for half-maximal stimulation (EC₅₀) for TRβ1 was 2×10⁻¹² M and for TRα1 it was about 10⁻¹⁰ M. The compound (3,5-dimethyl-4-(4′-hydroxy-3′-isopropylbenzyl)-phenoxy acetic acid, referred to as GC-1, showed approximately 10 times preference for binding TRβ1.⁴ Both of these compounds have methyl groups in place of iodines on the inner ring and the outer ring iodine has been replaced by an isopropyl group. Unlike T₃, in which the side chain is a three-carbon amino acid, the side chains of these analogs contain a nitrogen or oxygen linked with a carbonyl or methylene carbon prior to the terminal carboxylic acid. These structural features provide the analogs with greater affinity for TRβ1 and somewhat different pharmacological properties.
⁴Chiellini G, Apriletti J W, al Yoshihara H, Baxter J D, Ribeiro R C, Scanlan T S, 1998 A high-affinity subtype-selective agonist ligand for the thyroid hormone receptor. Chem Biol 5: 299-306.

CGS 23425 lowered cholesterol and LDL-cholesterol in fat-fed rats in parallel suggesting cholesterol reduction in these animals was primarily through receptor-mediated removal of LDL-cholesterol in the liver.⁵ LDL receptor number was increased in HepG2 cells treated with this compound. Comparison of equimolar doses of GC-1 with T₃ revealed GC-1 had similar lipid lowering effects without increasing heart rate. At higher doses the compounds caused similar increases in heart rate. GC-1 had some inotropic activity in hypothyroid animals but did not increase SR Ca²-ATPase mRNA or switch myosin isoforms.⁶ A recently reported TRβ1-selective analog, 3,5-dichloro-4[(4-hydroxy-3-isopyropylphenoxy)phenyl] acetic acid (KB-141), binds with 14 times greater affinity to TRβ1 than TRα1 and has been reported to have a 10-fold difference between heart rate increase and cholesterol-lowering activity.⁷ Study of a series of homologous carboxylic acid derivatives indicated that receptor binding increased with chain length in the order formic, acetic and propionic acid, while β1-selectivity was highest with the acetic acid side chain.
⁵Taylor A H, Stephan Z F, Steele R E, Wong N C W 1997 Beneficial effects of a novel thyromimetic on lipoprotein metabolism. Mol Pharmacol 52: 542-4735.

⁶Trost S U, Swanson E, Gloss B, Wang-Iverson D B, Zhang H, Volodarsky T. Grover G J, Baxter J D, Chiellini G, Scanlan T S, Dillmann W H. The thyroid hormone receptor-β-selective agonist GC-1 differentially affects plasma lipids and cardiac activity. Endocrinology 2000; 141: 3057-64.

⁷Ye L, Li Y L, Mellstrom K, Mellin C, Bladh L G, Koehler K, Garg N, Collazo G, Litten C, Husman B, Persson K, Ljunggreen J, Grover G, Sleph P G, Malm G R 2003. Thyroid receptor ligands, 1. Agonist ligands selective for the thyroid receptor beta1. J Med Chem 46: 1580-8.

Various other thyroid hormone analogs have been described in the patent literature. See, for example, U.S. Pat. No. 6,017,958, which describes various thyroid hormone analog compounds having the structural formula: 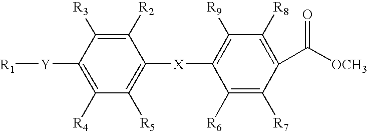
and pharmaceutically acceptable salts thereof, wherein:
X=O, S, CH₂, carboxy or absent;
Y=O or S;
R₁=methyl or ethyl;
   R₂, R₃, R₄ and R₅ are each independently selected from the group consisting of: H, (C₁-C₄) alkyl, (C₁-C₄) alkenyl, (C₁-C₄) alkynyl, hydroxy, (C₁-C₄) alkoxy and halogen; and
   R₆, R₇, R₈ and R₉ are each independently selected from the group consisting of: H, (C₁-C₄) alkyl, (C₁-C₄) alkenyl, (C₁-C₄) alkynyl, hydroxy, (C₁-C₄) alkoxy, halogen, NO₂ and NH₂.

Also described are compounds having the structural formula: 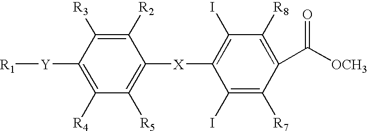
and pharmaceutically acceptable salts thereof, wherein:
X=O, S, CH₂, carboxy or absent;
Y=O or S;
R₁=methyl or ethyl;
   R₂, R₃, R₄ and R₅ are each independently selected from the group consisting of: H, (C₁-C₄) alkyl, (C₁-C₄) alkenyl, (C₁-C₄) alkynyl, hydroxy, (C₁-C₄) alkoxy and halogen; and

R₇ and R₈ are each independently selected from the group consisting of: H, (C₁-C₄) alkyl, (C₁-C₄) alkenyl, (C₁-C₄) alkynyl, hydroxy, (C₁-C₄) alkoxy, halogen, NO₂ and NH₂. Yet other thyroid hormone analogs described in the literature include DITPA as taught in my aforesaid U.S. Pat. No. 6,534,676, and TRIPROP and TETRAPROP as taught in my aforesaid co-pending application Ser. No. 10/368,755.

Prior to administration to either human patients, or to animals, the selected thyroid hormone analog may be dispersed or dissolved in a pharmaceutically acceptable carrier and, if desired, further compounded with one or more ingredients selected from a stabilizer, an excipient, a solubilizer, an antioxidant, a pain-alleviating agent, an isotonic agent, and combinations thereof.

The selected thyroid hormone analog may be formulated as a liquid preparation, e.g., for parenteral administration intravenously, subcutaneously or intramuscularly, or intranasally or orally, as a solid preparation for oral administration, e.g., pills, tablets, powders, or capsules, as an implant preparation, or as a suppository for rectal administration. For example, the formulation for parenteral administration for injection may be prepared by conventional methods known to a person skilled in the art, such as by dissolving the selected thyroid hormone analog in an appropriate solvent or carrier such as sterilized water, buffered solution, isotonic sodium chloride solution and the like, and may be formulated as solutions, emulsions or suspensions. For rectal administration, a unit dose of the selected thyroid hormone analog may be formulated with cocoa butter or a glyceride.

The selected thyroid hormone analog also may be administered in the form of inhalation or insufflation. For administration by inhalation or insufflation a solution of the selected thyroid hormone analog is conveniently delivered in the form of an aerosol spray presentation from pressurized packs or nebulizer, with the use of suitable propellants such as carbon dioxide or other suitable gasses. In addition, the selected thyroid hormone analog may be administered using other conventional drug delivery systems well known to a person skilled in the art. Examples of the preparations for drug delivery system are microspheres (nanoparticle, microparticle, microcapsule, bead, liposome, multiple emulsion, etc.) and the like.

A stabilizer may be added to the formulation, and the examples of a stabilizer include albumin, globulin, gelatin, mannitol, glucose, dextran, ethylene glycol and the like. The formulation of the present invention may include a necessary additive such as an excipient, a solubilizer, an antioxidant agent, a pain-alleviating agent, an isotonic agent and the like. The liquid formulation may be stored in frozen condition, or after removal of water by a process such as freeze-drying. The freeze-dried preparations are used by dissolving in pure water for injection and the like before use.

Selection of the specific thyroid hormone analog and of effective dosages and schedules for administering the selected thyroid hormone analog may be determined empirically by measuring for possible increase in cardiac output and monitoring for possible increase in heart rate. An administration route of the preparation may vary depending on the form of preparation. For example, the parenteral preparation may be administered intravenously, intraarterially, subcutaneously or intramuscularly.

The selected thyroid hormone analogs also may be formulated for transdermal or implant administration. Such long acting implantation administrations include subcutaneous or intramuscular implantation. Thus, for example, the selected thyroid hormone analog may be formulated with suitable polymeric or hydrophobic materials (for example as an emulsion in an acceptable oil) or ion exchange resins or as sparing soluble derivatives, for example as a sparingly soluble salt.

A suitable transdermal delivery system includes a carrier, such as a liquid, gel, solid matrix, or pressure sensitive adhesive, into which the selected thyroid hormone analog is incorporated. In one embodiment, no backing material is present. In an alternative embodiment, backing may be used in combination with a carrier. In this later embodiment, portions of the carrier that are not in physical contact with the skin or mucosa may be covered with a backing, which serves to protect the carrier and the components contained in the carrier, including the selected thyroid hormone analog being delivered, from the environment. Backings suitable for such use include metal foils, metalized plastic films, and single layered and multilayered polymeric films.

For transdermal delivery the selected thyroid hormone analog may be dissolved in a solvent system. A suitable solvent system may include water, and optionally one or more lower alcohols such as ethanol, isopropyl alcohol, propyl alcohol, and the like. Preferably, such alcohols have carbon contents between 2 and about 6. The solvent system may additionally include a glycol such as ethylene glycol, propylene glycol, glycerol, and the like. The solvent system also may include one or more dialkylsulfoxides and/or dialkylsulfones, and/or one or more ketones, ethers, and esters, such as acetone, methylethylketone, dimethylether, diethylether, dibutylether, and alkyl acetates, alkyl proprionates, alkyl butyrates, and the like.

Although solutions of the selected thyroid hormone analog are preferred, emulsions may be used. Such emulsions may be aqueous, wherein the aqueous phase is the major and continuous phase, or non-aqueous, wherein a water-insoluble solvent system comprises the continuous phase.

As with DITPA of my parent patent, the transdermal delivery of the selected thyroid hormone analog is effective to treat chronic heart failure and/or lower LDL-cholesterol levels even without including a substance capable of in vivo stimulation of adenosine 3′,5′-cyclic monophosphate, and even without including a substance capable of in vivo stimulation of guanosine 3′,5′-cyclic monophosphate. If desired, substances such as an extract of Coleus Forskholi, optionally may be included in the transdermal delivery of the selected thyroid hormone analog-containing formulations at a level of between about 0.0001 weight percent to about 1.0 weight percent.

The transdermal delivery the selected thyroid hormone analog-containing formulations also may contain agents known to accelerate the delivery of medicaments through the skin or mucosa of animals, including humans. These agents are sometimes known as penetration enhancers, accelerants, adjuvants, and sorption promoters, and are collectively referred to herein as “enhancers.” Some examples of enhancers include polyhydric alcohols such as dipropylene glycol; oils such as olive oil, squalene, and lanolin; polyethylene glycol ethers and fatty ethers such as cetyl ether and oleyl ether; fatty acid esters such as isopropyl myristate; fatty acid alcohols such as oleyl alcohol; urea and urea derivatives such as allantoin; polar solvents such as dimethyldecylphosphoxide, methyloctylsulfoxide, dimethylacetonide, dimethyllaurylamide, dodecylpyrrolidone, isosorbitol, decylmethylsulfoxide, and dimethylformamide; salicylic acid; benzyl nicotinate; bile salts; higher molecular weight aliphatic surfactants such as lauryl sulfate salts. Other agents include oleic acid and linoleic acids, ascorbic acid, panthenol, butylated hydroxytoluene, tocopherol, tocopheryl acetate, tocopheryl linoleate, propyloleate, isopropyl palmitate, oleamide, polyoxyethylene lauryl ether, polyoxyethylene olelyl ether and polyoxyethylene oleyl ether. In this embodiment, these skin penetration enhancers are present from about 0.01 weight percent to about 5 weight percent.

The transdermal formulations delivery system can be prepared using conventional methods to apply an appropriate carrier to an appropriate backing. For example, a device can be prepared by preparing a coating formulation by mixing a solution of the adhesive in a solvent system containing the selected thyroid hormone analog, and any other desired components, to form a homogeneous solution or suspension; applying the formulation to a substrate such as a backing or a release liner; using well known knife or bar or extrusion die coating methods; drying the coated substrate to remove the solvent; and laminating the exposed surface to a release liner or backing.

The following example further illustrates the present invention.

### EXAMPLE 1

After an initial safety study in 7 normal volunteers, a randomized double-blind comparison was made between 3,5-diiodothyropropionic acid (DITPA) made in accordance with my aforesaid U.S. Pat. No. 6,534,676 and placebo in 19 patients with moderately severe congestive failure. In heart failure patients receiving the drug for 4 weeks, cardiac index was increased (p=0.04) and systemic vascular resistance index was decreased (p=0.02). Systolic cardiac function was unchanged but isovolumetric relaxation time was decreased significantly, suggesting improvement in diastolic function. Total serum cholesterol (p=0.005) and triglycerides (p=0.01) also were decreased significantly.

The results are summarized and tabulated below:

| | Baseline | 4 weeks |
|---|---|---|
| Heart Rate | 81.3 ± 4.4 | 82.2 ± 4.5 |
| Cardiac Output | 4.5 ± 0.36 | 5.3 ± 0.36 (P < 0.05) |
| Cardiac Index | 2.1 ± 0.14 | 2.5 ± 0.16 (P < 0.05) |
| Mean Arterial Pressure | 96.2 ± 6.68 | 92.0 ± 5.74 |
| SVRI | 3465.3 ± 319.4 | 2643.6 ± 256.7 (P < 0.05) |

SVRI = Systemic Vascular Resistance Index

While the invention has been described in detail herein in accordance with certain preferred embodiments thereof, many modifications and changes therein may be effected by those skilled in the art. Accordingly, it is intended by the appended claims to cover all such modifications and changes as fall within the true spirit and scope of the invention.

### EXAMPLE 2

An experimental study was carried out in the rabbit postinfarction model of heart failure as described in my aforesaid U.S. Pat. No. 6,534,676. Infarction resulted in increased LV end-diastolic pressure (EDP) and prolonged the time constant for LV relaxation (τ) (p=0.001 for both variables). Postinfarction treatment with DITPA for 3 weeks decreased LV EDP and increased the rate of increase in LV pressure (+dP/dt), a measure of myocardial contractility. The time constant of LV relaxation (τ) also was decreased. Because of the faster baseline heart rate in this species the numerical increase after treatment was greater than 10 beats per minute but the percentage increase was only 5%, which was not statistically significant (p=0.5). The improvement in LV contractility and relaxation are equivalent to the improvement in cardiac output in example 1. The results are summarized and tabulated below:

| | Infarcted | Treated |
|---|---|---|
| Heart Rate, bpm | 265 ± 32 | 278 ± 13 (P = 0.5) |
| +dP/dt, mmHg/sec | 4782 ± 1130 | 6239 ± 844 (P = 0.002) |
| τ, millisec | 21.1 ± 3.9 | 14.9 ± 1.5 (P = 0.001) |

Values are mean ± SD for 13 infarcted control animals and 9 infarcted animals treated with DITPA for 3 weeks.

Mahaffey K. W., Raya T. E., Pennock G. D., Morkin E., Goldman S.: Left Ventricular Performance and Remodeling in Rabbits after Myocardial Infarction. Circulation 91: 794-802, 1995.

1. A method for treatment of a patient with congestive heart failure, comprising administering to the patient a therapeutically effective amount of a thyroid hormone analog sufficient to produce an increase in cardiac index of at least 15% while increasing heart rate no more than 10 beats per minute.

2. The method of claim 1, wherein the thyroid hormone analog is administered as a formulation selected from the group consisting of a liquid preparation, solid preparation, capsule preparation, and an implant preparation.

3. The method of claim 2, wherein said formulation further comprises a pharmaceutically acceptable carrier.

4. The method of claim 3, wherein said formulation further comprises at least one of a stabilizer, excipient, solubilizer, antioxidant, pain-alleviating agent, and an isotonic agent.

5. The method of claim 1, wherein the thyroid hormone analog is administered by parenteral injection.

6. The method of claim 5, wherein the thyroid hormone analog is administered by parenteral intravenous injection.

7. The method of claim 1, wherein the thyroid hormone analog is administered orally.

8. The method of claim 1, wherein the thyroid hormone analog is administered directly to the pulmonary system of the patient.

9. The method of claim 1, wherein the thyroid hormone analog is administered transdermally.

10. The method of claim 1, wherein the thyroid hormone analog is administered by implantation.
